(19) United States
(12) Patent Application Publication
(10) Pub. No.: US 2018/0100142 A1
(43) Pub. Date: Apr. 12, 2018

(54) Methods and Kits for Production of Tissue Equivalents from Cryopreserved Cells

(71) Applicant: MatTek Corporation, Ashland, MA (US)

(72) Inventors: Patrick J. Hayden, Ashland, MA (US); Michael Bachelor, Worcestor, MA (US); Mitchell Klausner, Sharon, MA (US)

(73) Assignee: MatTek Corporation, Ashland, MA (US)

(21) Appl. No.: 15/287,690

(22) Filed: Oct. 6, 2016

(51) Int. Cl. C12N 5/071

(52) U.S. Cl. CPC C12N 5/0698; C12N 5/0688; C12N 5/0629; C12N 5/0626; C12N 2533/90; C12N 2533/70; C12N 2533/54; C12N 2501/15; C12N 2501/11; C12N 2533/50; C12N 2533/52

(57) ABSTRACT

Methods are provided for production of differentiated tissue equivalent model systems from cryopreserved undifferentiated cells. The methods include seeding undifferentiated cells directly onto a support for differentiation immediately after recovery from cryopreservation, without intervening steps for expansion, harvesting, counting, or dilution of the cells.

This invention was made with government support under SBIR Project 5R44GM084551-03, awarded by the National Institutes of Health. The government has certain rights in the invention.

### FIELD OF THE INVENTION

The invention relates to methods for production of tissue equivalent model systems, in particular methods that include direct seeding of cryopreserved cells onto a support for differentiation.

### BACKGROUND

In vitro tissue model systems, or “tissue equivalents,” may be used to study the effects of various agents on a variety of cell types. For example, in vitro skin equivalents may be used to test the effects of substances such as cosmetics or medications, or agents such as light and heat, for potential toxicity, irritation, or inflammation that may result from their application to skin. In comparison with in vivo animal models or studies using human test subjects, such tissue model systems offer substantial advantages in terms of reproducibility, speed of testing, and reduced cost.

Traditional methods for production of three-dimensional tissue models require significant expenditures of time and labor. Cells from each donor must be expanded, harvested, counted, and diluted to an appropriate concentration prior to seeding the cells onto tissue culture inserts. The need to perform each of these tasks every time a new batch of tissues is produced is problematic due to significant variability in each step of the process. This results in variability in the end product, and can lead to delays or loss of production if there is a failure in any of these tasks. Producing three-dimensional tissue models from multiple donors at the same time exacerbates these problems because each step must be duplicated for each donor. There is a need for a more streamlined process for producing tissue models, with a reduction in labor required and variability of the end product.

State of the art methods of 3D tissue model production, as reported in the current literature, invariably include the steps of: 1) propagation of cells in monolayer culture to produce a large number of cells (i.e., cell expansion); 2) harvesting the monolayer cells from the culture vessels using enzymes to release the cells from the culture vessels; 3) counting and diluting the cells, followed by seeding the cells onto tissue culture inserts. (FIG. 1) Examples include protocols published in the literature by recognized experts in the field (Fulcher, M. L., et al. (2005) Methods Mol Med 107:183-206; Li, L., Fukunaga-Kalabis, M., Herlyn, M. The Three-Dimensional Human Skin Reconstruct Model: a Tool to Study Normal Skin and Melanoma Progression. J. Vis. Exp. (54), e2937, doi:10.3791/2937 (2011); Egles C, Garlick J A, Shamis Y. Three-Dimensional Human Tissue Models of Wounded Skin. Methods in molecular biology (Clifton, N.J.). 2010; 585:345-359. doi:10.1007/978-1-60761-380-0_24).

U.S. Pat. No. 8,222,031 B2, and use protocols provided by vendors of commercial cells, culture media and related products that are commonly utilized to produce 3D tissue models. Protocols provided by leading commercial vendors of human cells and/or culture media explicitly teach that the cells must be expanded immediately prior to seeding, and stress that direct seeding onto inserts immediately after thawing should not be done (Lonza, Document #INST-CC-4539 09/11 (2011) (http://bio. lonza.com/uploads/tx_mwaxmarketingmaterial/Lonza_ManualsProductInstructions_Instructions_-_S-ALI Air-Liquid_Interface_Medium.pdf); PneumaCult: An integrated culture medium system for in vitro human airway modeling (2014) (https://www.stemcell.com/media/files/poster/SP00118-PneumaCult_an_Integrated_Culture_Medium_System_Vitro_Human_Airway_Modeling.pdf); Lifeline Tech Air Liquid Interface-BTE Protocol d4 (http://www.lifelinecelltech. com/pdf/INM%20BTEC-ALI%200414%20v2%20LM-0049_50.pdfi.

### BRIEF SUMMARY OF THE INVENTION

Methods are provided for production of 3D tissue models directly from cryopreserved cells without the need for prior expansion. Cells may be cryopreserved at a high density, so that 1 vial of cells can seed a large number of tissues. (FIG. 2) Several cell types (e.g., keratinocytes and melanocytes) may be mixed together prior to cryopreservation. After thawing, cells are diluted in a small volume of culture medium and then separated from the medium (e.g., centrifuged). The separated cells (e.g., cell pellet) are resuspended in a volume of medium appropriate for the number of desired supports (e.g., inserts), and the cells are then directly seeded onto the supports without the additional expansion that is described in the literature.

This new process is more reliable, more reproducible, saves labor and time and avoids the need for duplication of tasks in order to manufacture single cell type 3D tissue models and co-culture models, such as 3D tissue models that contain melanocytes or immune system cells. The new process also allows multiple donors, reporter models, or cell models to be conveniently seeded onto the same multiwell plate (e.g., 96-well or 24-well high throughput plates). “Do-it-yourself” 3D model kits are also provided that will permit construction of 3D tissue models utilizing the direct seed method. Existing commercial do-it-yourself 3D model kits require cells to be expanded prior to use (Lonza; PneumaCult; Lifeline; supra)

Cells can be cryopreserved in a variety of densities or in any container sizes that are convenient for the desired size and number of 3D tissues to be produced. Many different types of cells may be used simultaneously including, but not limited to, keratinocytes, melanocytes, fibroblasts, endothelial cells, airway epithelial cells, corneal epithelial cells, vaginal epithelial cells, oral epithelial, and intestinal epithelial cells, amongst others.

In one aspect, a method is provided for producing a tissue equivalent. The method includes: (a) seeding frozen cryopreserved undifferentiated cells directly onto a support immediately following recovery from cryopreservation; and (b) culturing the cells in contact with a differentiation medium and under conditions appropriate for differentiation. In some embodiments, the differentiation medium includes at least about 200 μM calcium or greater than about 200 μM calcium and growth and differentiation factors for differentiation of the cells into a desired tissue. In some embodiments, recovery from cryopreservation includes rapidly thawing the cells and diluting the thawed cell suspension into culture medium, for example, seeding, propagation, or differentiation medium.

In some embodiments, between steps (a) and (b), the cells are cultured in contact with a propagation medium and under conditions appropriate for propagation, and the propagation medium is removed prior to addition of the differentiation medium in step (b). In some embodiments, the propagation medium includes less than about 200 μM calcium and growth factors and other components for propagation of the undifferentiated cells. In some embodiments, the cells are incubated in contact with the propagation medium for about 2 hours to about 24 hours or about 2, 3, 4, 5, 6, or 7 days to achieve confluence of cell monolayer prior to removal and replacement of the propagation medium with differentiation medium.

In some embodiments, the cells differentiate after contact with the differentiation medium for about 7 days to about 8, 9, 10, 11, 12, 13, 14, 15, 16, 17, 18, 19, 20, 21, 22, 23, 24, 25, 26, 27, or 28 days.

The cryopreserved cells may be at a density of about 1×10⁶ to about 20×10⁶ per mL, or about 1×10⁶ to about 5×10⁶ per mL, about 3×10⁶ to about 8×10⁶ per mL, about 5×10⁶ to about 10×10⁶ per mL, about 8×10⁶ to about 13×10⁶ per mL, about 10×10⁶ to about 15×10⁶ per mL, about 13×10⁶ to about 18×10⁶ per mL, about 15×10⁶ to about 20×10⁶ per mL, or about 18×10⁶ to about 20×10⁶ per mL in cryopreservation medium, prior to recovery from cryopreservation. In some embodiments, the cryopreservation medium includes one or more of serum, bovine serum albumin (BSA), glycerol, and dimethylsulfoxide (DMSO). In some embodiments, the cryopreservation medium includes a pH of about 6.8 to about 7.8. In some embodiments, the cryopreservation medium includes osmolality of about 250 to about 350 mOsm/kg.

After recovery from cryopreservation, the cryopreserved cells are seeded directly onto the support, for example, at a density of about 100×10³ to about 200, 300, 400, 500, 600, 700, 800, 900, 1000, 1100, 1200, 1300, 1400, or 1500×10³ per cm².

In some embodiments, the support includes a microporous membrane through which culture medium may pass. In some embodiments, the membrane may be coated with extracellular matrix material. For example, the extracellular matrix material may include one or more of an attachment factor, a cell surface integrin, collagen I, III, or IV, fibronectin, laminin, hyaluronic acid, and a growth factor. In some embodiments, the attachment factor includes one or more of laminin, entactin, collagen, and heparin sulfate proteoglycans. In one embodiment, the attachment factor includes Matrigel®. In some embodiments, the growth factor includes transforming growth factor (TGF)-beta and/or epidermal growth factor (EGF). The membrane may include, for example, polycarbonate, polyester, modified polytetrafluorethylene, microporous silica, ceramic material, or cellulose nitrate.

In some embodiments, the support is in the form of an insert that fits within a well of a multiwell plate. In some embodiments, the support is in the form of a high throughput screening (HTS) plate, e.g., with the microporous membrane in the bottoms of wells of the plate.

In some embodiments, the cells include one or more of undifferentiated keratinocytes, melanocytes, fibroblasts, endothelial cells, airway epithelial cells, corneal epithelial cells, vaginal epithelial cells, oral epithelial cells, intestinal epithelial cells, dendritic cells, macrophages, kidney cells, liver cells, neuronal cells, and mast cells. In one embodiment, the cells include undifferentiated skin cells, for example, keratinocytes, melanocytes, or a mixture of keratinocytes and melanocytes. In an embodiment, the undifferentiated skin cells include a mixture of keratinocytes and melanocytes in a ratio of about 3:1 to about 30:1. In some embodiments, the cells include human cells. In some embodiments, the cells include non-human cells, for example, non-human mammalian cells.

In another aspect, a kit is provided for producing a tissue equivalent. The kit includes, for example: (a) frozen cryopreserved undifferentiated cells; (b) a support for growth of the cells; and (c) one or more culture media for propagation and/or differentiation of the cells. In some embodiments, the kit further includes instructions directing seeding of the cells directly onto the support immediately following recovery from cryopreservation, without additional steps for expansion, harvesting, counting, and/or dilution of the cells beforehand.

In some embodiments, the cryopreserved cells in the kit include a density of about 1×10⁶ to about 20×10⁶ per mL, or about 1×10⁶ to about 5×10⁶ per mL, about 3×10⁶ to about 8×10⁶ per mL, about 5×10⁶ to about 10×10⁶ per mL, about 8×10⁶ to about 13×10⁶ per mL, about 10×10⁶ to about 15×10⁶ per mL, about 13×10⁶ to about 18×10⁶ per mL, about 15×10⁶ to about 20×10⁶ per mL, or about 18×10⁶to about 20×10⁶ per mL, in cryopreservation medium. In some embodiments, the cryopreservation medium includes one or more of serum, BSA, glycerol, and DMSO.

In some embodiments, the support in the kit includes a microporous membrane through which culture medium may pass. The membrane may be coated with extracellular matrix material, including, for example, but not limited to, one or more of an attachment factor, a cell surface integrin, collagen I, III, or IV, fibronectin, laminin, hyaluronic acid, and a growth factor. The support may be in the form of an insert that fits within a well of a multiwell plate or in the form of a HTS plate. The membrane may include, for example, but not limited to, polycarbonate, polyester, modified polytetrafluorethylene, microporous silica, ceramic material, or cellulose nitrate.

In some embodiments, the undifferentiated cells in the kit include one or more of, for example, but not limited to, undifferentiated keratinocytes, melanocytes, fibroblasts, endothelial cells, airway epithelial cells, corneal epithelial cells, vaginal epithelial cells, oral epithelial cells, intestinal epithelial cells, dendritic cells, macrophages, kidney cells, liver cells, neuronal cells, and mast cells. In some embodiments, the cells include undifferentiated skin cells, for example, keratinocytes, melanocytes, or a mixture of keratinocytes and melanocytes. In some embodiments, the undifferentiated cells are undifferentiated human cells. In some embodiments, the undifferentiated cells are non-human cells, for example, non-human mammalian cells.

### BRIEF DESCRIPTION OF THE DRAWINGS

FIG. 1 depicts a standard method in the art for 3D tissue model production.

FIG. 2 depicts an embodiment of a direct seed method for 3D tissue model production as disclosed herein.

FIG. 3 shows hematoxylin and eosin (H&E) stained images (300× Magnification) of EpiDerm™, EPI-200 (Standard Method) and EPI-200 (Direct Seed) tissues prepared as described in Example 1.

FIG. 4 shows macroscopic pigmentation of MelanoDerm™, MEL-300-B, prepared by the Standard Method (Lot 21895) and MEL-300-B, prepared by the Direct Seed method (Lot 21897). Following attainment of full differentiation as described in Example 2, pigmentation was induced from 0-14 days in culture using two types of medium (EPI-100-NMM-13 and EPI-100-LLMM).

FIG. 5 shows H&E stained images (300× Magnification) of EpiAirway™, AIR-100 produced by the Standard Method (AR5176) and Direct Seed method (AR5729), as described in Example 3.

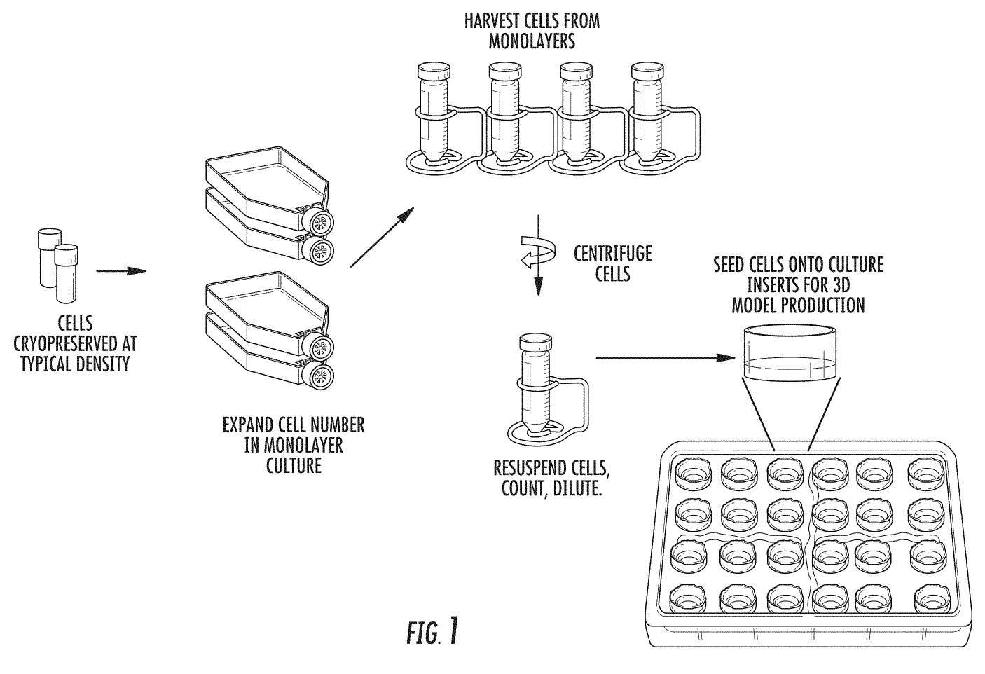

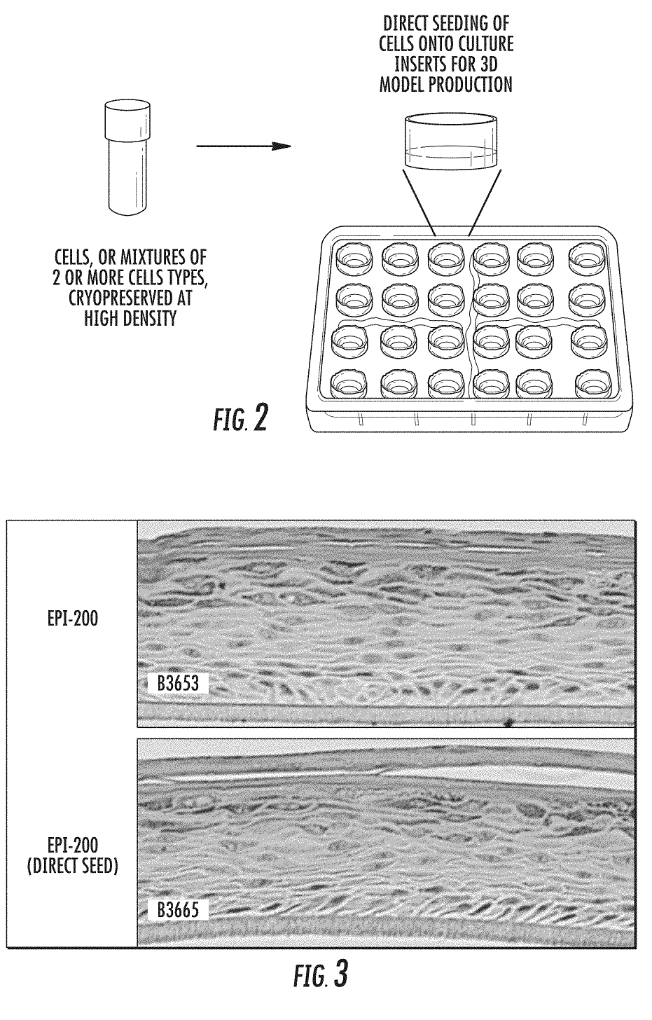

### DETAILED DESCRIPTION

Methods and kits for producing differentiated tissue equivalents are provided herein. The disclosed methods include direct seeding of cryopreserved undifferentiated cells onto a support for differentiation, versus methods that are standard in the art that include one or more additional step(s) prior to seeding onto the support.

Unless defined otherwise herein, all technical and scientific terms used herein have the same meaning as commonly understood by one of ordinary skill in the art to which this invention belongs. Singleton, et al., Dictionary of Microbiology and Molecular Biology, second ed., John Wiley and Sons, N.Y. (1994), and Hale & Markham, The Harper Collins Dictionary of Biology, Harper Perennial, N.Y. (1991) provide one of skill with a general dictionary of many of the terms used in this invention. Any methods and materials similar or equivalent to those described herein can be used in the practice or testing of the present invention.

The practice of the present invention will employ, unless otherwise indicated, conventional techniques of molecular biology (including recombinant techniques), microbiology, cell biology, and biochemistry, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); and Gene Transfer and Expression: A Laboratory Manual (Kriegler, 1990).

Numeric ranges provided herein are inclusive of the numbers defining the range.

“A,” “an” and “the” include plural references unless the context clearly dictates otherwise.

### Methods for Producing Tissue Equivalents

Methods are provided for production of a differentiated tissue equivalent, such as a three-dimensional tissue equivalent, from cryopreserved undifferentiated cells. The methods disclosed herein include seeding frozen cryopreserved cells directly onto a support immediately following recovery from cryopreservation, i.e., without intervening step(s) that are standard in the art, such as expansion, harvesting, counting, and/or dilution of the cells prior to seeding. The seeded cells are cultured in contact with a differentiation medium under conditions that are suitable for differentiation into a tissue of interest. Optionally, the cells are cultured in a propagation medium after seeding on the support and prior to addition of differentiation medium, for example, until a confluent cell monolayer is achieved.

The undifferentiated cryopreserved cells may be of a single cell type, or may include two or more cell types for co-culture to produce a tissue equivalent that includes multiple differentiated cell types.

In some embodiments, the cryopreserved undifferentiated cells are provided in a volume of about 1 mL to about 5 mL at a density of about 6×10⁶ to about 20×10⁶ per mL. Recovery from cryopreservation may include, for example, rapidly warming the frozen cells, for example, at about 37° C. The cells may then be diluted into a culture medium and seeded directly onto a microporous support. For example, the cells may be seeded at a density of about 100×10³ to about 200×10³ per cm².

Recovery from cryopreservation may include rapidly thawing the cells and diluting the thawed cell suspension into culture medium, (e.g., seeding medium) in a concentration desired for seeding. In some embodiments, the cell suspension may be centrifuged to pellet the cells, followed by re-suspension of the cells in fresh culture medium (e.g., seeding medium) in order to remove cryopreservation medium components (e.g., DMSO or glycerol) prior to seeding.

Cells may be grown until confluent, for example, in propagation medium, and then differentiated in differentiation medium. In some embodiments, the cells incubated in propagation medium for about 2 hours to about 7 days to achieve confluence of cell monolayer prior to removal of the propagation medium and replacement with differentiation medium. In some embodiments, after addition of the differentiation medium, the cells differentiate in about 7 days to about 28 days to produce a differentiated tissue equivalent.

### Media

Media used for cryopreservation of undifferentiated cells that are seeded directly onto a support immediately after recovery from cryopreservation may include serum, bovine serum albumin (BSA), DMSO, and/or glycerol. In some embodiments, the pH of cryopreservation media is about 6.8 to about 7.8. In some embodiments, osmolality is controlled by, for example, by modifying the salt concentration. In some embodiments, osmolality in the cryopreservation media is about 250 to about 350 mOsm/kg. Undifferentiated cells are preserved in cryopreservation media using methods that are well known in the art, for example, as described in Thermo Scientific Tech Note No. 14, https://static.thermoscientific.com/images/D19575˜.pdf.

In some embodiments, a “seeding medium” may be used for seeding cells that have been recovered from cryopreservation onto a support. “Seeding medium” may include supplements to promote cell attachment, such as, but not limited to, one or more extracellular matrix component and/or cell attachment factor, e.g., fibronectin, laminin, collagen type I, type III, or type IV. In some embodiments, seeding medium contains growth factors and supplements as described herein for propagation or differentiation medium but in addition may contain one or more extracellular matrix component and/or cell attachment factor.

The culture media used for propagation and differentiation of the cells into a three-dimensional tissue equivalent, as described herein, influence the properties of the final tissue equivalent product. Unless otherwise stated, the term “medium” as used herein is meant to include both serum containing and serum free medium. “Serum free medium” refers to medium which does not contain serum or a fractionated portion thereof. All components and amounts of serum free medium, in terms of their chemical compositions, are defined and relatively pure by tissue culture standards of the art.

The term “differentiation medium” is used herein to refer to culture medium used for transforming the cells seeded onto the microporous membrane or some other culture support into a 3-dimensional tissue equivalent. This may involve growing the cells at the air-liquid interface, submerged in differentiation medium, or growing the cells using the wet film technique (in which a small amount of differentiation medium is added to the apical surface). The purpose of this medium is to induce the cells to organize into an in vitro tissue which mimics the in vivo tissue in structure and function. Differentiation medium may also be used to maintain the tissue in a differentiated state for an extended period of time. In some embodiments, differentiation medium includes about 0.2 mM to about 2.0 mM calcium, about 0.2 mM to about 0.5 mM, about 0.3 mM to about 0.8 mM, about 0.5 mM to about 1.0 mM, about 0.8 mM to about 1.3 mM, about 1.0 mM to about 1.5 mM, about 1.3 mM to about 1.8 mM, or about, 1.5 mM to about 2.0 mM, or any of about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mM calcium.

A variety of cell culture media known in the art are suitable for use as differentiation medium as described herein, the determination of which is within the ability of one of average skill in the art. In one embodiment, the differentiation medium comprises a retinoid, such as retinoic acid, retinol, retinyl acetate, 13-cis retinoic acid, or 9-cis retinoic acid. In an embodiment, the medium comprises about 10⁻⁵ to about 10⁻¹³ M of the retinoid (e.g., about 5×10⁻¹⁰ M of a retinoid such as retinoic acid). In one embodiment, the concentration of the retinoid is reduced incrementally over the period of culture. For example, the level of retinoic acid may be reduced from about 5×10⁻⁹ M down to about 5×10⁻¹³ M over the course of the differentiation period.

In a preferred embodiment, the differentiation medium contains one or more of the following supplements: adenine, a-melanocyte stimulating hormone, arachidonic acid, β-fibroblast growth factor, bovine pituitary extract, bovine serum albumin, calcium chloride, calf serum, carnitine, cholera toxin, dibutyl cyclic adenosine monophosphate, endothelin-1, EGF (epidermal growth factor), epinephrine, estradiol, estrogen, ethanolamine, fetal bovine serum, FLT-3 (Fms-like tyrosine kinase 3), glucagon, granulocyte/macrophage-colony stimulating factor, hepatocyte growth factor, horse serum, human serum, hydrocortisone, insulin, insulin-like growth factor 1, insulin-like growth factor 2, interleukin-1β, interleukin-3, interleukin-4, interleukin-6, interleukin-12, interleukin-18, iso-butyl methyl xanthine, isoproterenol, keratinocyte growth factor, linoleic acid, MIP-1α (macrophage inflammatory protein-1α), MIP-3a (macrophage inflammatory protein-3α), newborn calf serum, nor-epinephrine, oleic acid, palmitic acid, phosphoethanolamine, progesterone, stem cell factor, transferrin, transforming growth factor-β1, triidothyronine, tumor necrosis factor a, vitamin A, vitamin B12, vitamin C, vitamin D, and vitamin E.

In one embodiment, the differentiation medium contains: about a 3:1 ratio of DMEM:Ham's F12, about 10% fetal calf serum, about 10 ng/ml epidermal growth factor, about 0.4 μg/ml hydrocortisone, about 1×10⁻⁶ M isoproterenol, about 5 μg/ml transferrin, about 2×10⁻⁹ M triiodothyronine, about 1.8×10⁻⁴ M adenine, about 5 μg/ml insulin, and about 1×10⁻⁶ M retinoic acid.

In another embodiment, the differentiation medium is serum free. Serum free medium may be made using basic media or components known in the art (e.g., DMEM (Dulbecco's Modified Eagle's Medium), PRGM (Prostate Epithelial Cell Growth Medium Part #CC-3166, Biowhittaker, Inc.), Ham's F12 medium, MEM (Modified essential medium), McCoy's 5A medium, MCDB 153 (Molecular Cell and Developmental Biology 153 medium) KGM (Keratinocyte growth Medium, Biowhittaker) EpiLife (Cascade Biologics, Inc.), or Medium 199). In one embodiment, the serum free medium is about a 3:1 ratio of DMEM:F12, supplemented with additional defined (non-serum) components, such as retinoic acid, or any of the other defined components described herein.

In an embodiment, the serum free differentiation medium contains about a 3:1 ratio of DMEM:F12, about 5×10⁻¹⁰ M retinoic acid, about 0.3 ng/ml keratinocyte growth factor, about 5 ng/ml EGF, about 0.4 μg/ml hydrocortisone, and about 5 μg/ml insulin. In another embodiment, the serum free differentiation medium is DMEM:F12 (about 3:1 ratio) containing retinoic acid (RA) at about 5×10⁻⁹ M, keratinocyte growth factor (KGF) at about 0.1 nM, about 0.4 μg/ml hydrocortisone, about 5 μg/ml insulin, SCF (about 2.5 ng/ml), GM-CSF (about 20 U/ml), TNF-α (about 0.25 ng/ml), and FLT-3 (about 2 ng/ml).

A variety of cell culture media known in the art is suitable for use as growth medium for cultivation of cells, for example, in submerged medium. The term “propagation medium” or “growth medium” or “proliferation medium” is used herein to refer to medium used for growth of the cells prior to differentiation. In some embodiments, propagation medium includes about 20 μM to about 200 μM calcium, about 20 μM to about 50 μM, about 30 μM to about 80 μM, about 50 μM to about 100 μM, about 130 μM to about 180 μM, about 150 μM to about 190 μM, about 150 μM to about 200 μM, or any of about 20, 30, 40, 50, 60, 70, 80, 90, 100, 110, 120, 130, 140, 150, 160, 170, 180, 190, 195, or 200 μM calcium.

“Propagation medium” or “growth medium” or “proliferation medium,” as the terms are used herein, refer to culture medium that may or may not include supplements which induce or support differentiation of cells, and therefore the use of the term is not restricted to use for cellular propagation absent any level of differentiation of the cells. Examples of such medium include, without limitation, DMEM, PRGM, SFEM (serum free expansion medium), MEM, Medium 199, KGM, EpiLife, MCDB 153, McCoy's 5A. In some embodiments, the cells are grown submerged in propagation medium. However, the cells may alternatively be grown at the air-liquid interface or using the wet film technique (in which a small amount of propagation medium is added to the apical surface).

In one embodiment, the growth medium for cultivation of the cells is serum free. One example of serum free growth medium which can be used is (PRGM) containing SCF (about 25 ng/ml), GM-CSF (about 200 U/ml), TNF-α (about 2.5 ng/ml), and FLT-3 (about 20 ng/ml).

The determination of useful concentrations and combinations of the defined medium components or supplements described herein for use as growth medium or differentiation medium are within the ability of one of average skill in the art through no more than routine experimentation, as is the identification of additional supplements or medium components.

### Support

In some embodiments, the support onto which undifferentiated cells are seeded following recovery from cryopreservation medium, without intervening step(s) between recovery and seeding, such as cell expansion step(s), is in the form of a cell culture insert, a variety of which are known and available to the skilled artisan. The skilled artisan can envision additional receptacles, both with and without walls, which will suffice for use in the present method, all of which are intended to be encompassed by the present invention. If the receptacle has walls, the walls of the receptacle may consist of polystyrene, polycarbonate, resin, polypropylene, or other biocompatible plastic, with a porous base that serves as a support for the cells to adhere and develop. The porous base or support must allow for passage of media from underneath the developing tissue. The porous base may be a membranous base of polycarbonate or other culture compatible porous membrane such as membranes made of collagen, wettable fluoropolymers, cellulose, glass fiber or nylon attached to the bottom, on which the cells can be cultured. Examples of other suitable supports include, without limitation, an artificial membrane, an extracellular matrix component, a collagen gel, mixture or lattice, in vivo derived connective tissue, a mixed collagen fibroblast lattice, mixed extracellular matrix-fibroblast lattice, plastic, and a collagen sponge (Morota et al., (2000) U.S. Pat. No. 6,051,425). The support porosity must be of sufficient size to allow for passage of media, and can be readily determined by the skilled practitioner. In one embodiment, the average pore size may range from 0.1 μm to about 10 μm. The porous membrane may also be overlain with one of the other supports described herein. Preferably, the support components facilitate cellular attachment and development of the tissue.

In some embodiments, the support is in the form of an insert, e.g., a cylinder or other shaped enclosure with a microporous membrane at the bottom, with pores of a size through which culture medium may pass, wherein the insert is of appropriate shape and dimensions to fit within a well of a multiwell plate. In various embodiments, the multiwell plate is a 6-well plate (e.g., about 17.7 mL/well), a 12-well plate (e.g., about 6.8 mL/well), a 24-well plate (e.g., about 3.5 mL/well), a 48-well plate (e.g., about 1.6 mL/well), or a 96-well plate (e.g., about 0.3 mL/well). However, other formats, well volumes, and plate configurations may be used for the methods described herein.

In some embodiments, the support is in the form of a high throughput screening (HTS) plate, for example, containing 2-384 wells. The wells may have a microporous membrane bottom and the HTS plate may fit into an underplate reservoir which is configured to contain culture medium (e.g., differentiation medium) for culturing cells within the wells.

The membrane material may include polycarbonate, polyester, modified polytetrafluorethylene, microporous silica, ceramic material, and/or cellulose nitrate.

In some embodiments, the membrane is coated with an extracellular matrix material. For example, the extracellular matrix material may include one or more substance(s) including, but not limited to, an attachment factor, a cell surface integrin, collagen I, III, or IV, fibronectin, laminin, hyaluronic acid, and a growth factor. In some embodiments, the extracellular matrix material includes laminin, entactin, collagen, and/or heparin sulfate proteoglycans. In some embodiments, the extracellular matrix material includes TGF-beta and/or EGF.

### Cells

Any undifferentiated cells, e.g., undifferentiated mammalian cells, that may be cryopreserved and then recovered from cryopreservation and cultured in a differentiation medium to produce a tissue equivalent, e.g., a three-dimensional tissue equivalent, may be used in the methods described herein. A single cell type may be cultured or multiple cell types may be co-cultured to produce a tissue of interest. In some embodiments, the undifferentiated cells are human cells. In other embodiments, the undifferentiated cells are from rodents (e.g., mice, rats), dogs, cats, pigs, or monkeys or other non-human primates.

Nonlimiting examples of undifferentiated cell types that may be used in the methods described herein include keratinocytes, melanocytes, fibroblasts, endothelial cells, airway epithelial cells, corneal epithelial cells, vaginal epithelial cells, oral epithelial cells, intestinal epithelial cells, dendritic cells, macrophages, kidney cells, liver cells, neuronal cells (e.g., neurons, astrocytes, or other neuronal cells), and mast cells, and combinations thereof

In some embodiments, the cells are undifferentiated skin cells. For example, the cells may be keratinocytes, melanocytes, or a mixture of keratinocytes and melanocytes. In one embodiment, the cells are a mixture of keratinocytes and melanocytes in a ratio of about 3:1 to about 30:1.

### Kits

Kits are provided for producing tissue equivalents as described herein. A kit is provided whereby high density cryopreserved cells may be seeded directly onto a support for production of the tissue model without intervening steps such as cell expansion prior to seeding. For example, a kit may include: (a) frozen cryopreserved undifferentiated cells, as described herein; (b) a support for growth of the cells, as described herein; and (c) one or more culture media for propagation and/or differentiation of the cells, as described herein.

The kit may further include instructions directing seeing of the cells directly onto the support immediately following recovery, i.e., without intervening step(s) such as cell expansion, harvesting, counting, and/or dilution step(s) prior to seeding on the support. Instructions may be provided in printed form or in the form of an electronic medium such as a CD or DVD, or in the form of a website address where such instructions may be obtained.

The cryopreserved cells may be provided at a high density, since they do not require expansion prior to use. In some embodiments, the cryopreserved cells are provided at a density of about 6×10⁶ to about 10×10⁶ per mL in cryopreservation medium, or about 10×10⁶ to about 20×10⁶ per mL in cryopreservation medium.

### Methods of Use

Differentiated tissue equivalents that are produced using the methods disclosed herein may be used in a variety of screening and diagnostic assays. For example, the tissue equivalents may be used for testing of pharmacological and/or cosmetic effects of an agent on a tissue. The tissue equivalents may be used for testing or assaying one or more pharmacological, toxicological, physiological, morphological, inflammatory, and/or molecular biological parameter(s) effected by direct or indirect contact of an agent with the tissue. The effect(s) of an agent may be evaluated, for example, by histological, immunological, biochemical, and/or molecular biological methods. An “agent” may be a chemical compound, a biological substance, or an environmental parameter, such as heat or light.

The following examples are intended to illustrate, but not limit, the invention.

### EXAMPLES

### Example 1

### Production of 3D organotypic Skin Model Containing keratinocytes

Normal human epidermal keratinocytes were cryopreserved at a density of 8×10⁶ cells/mL in medium consisting of 80% (v/v) Medium 154 (Life Technologies), 10% (v/v) fetal bovine serum (Hyclone) and 10% (v/v) dimethylsulfoxide (Sigma) using a controlled rate freezer (Cryomed). Cells were stored in the vapor phase of a liquid N₂ storage dewar.

Cells were recovered from cryopreservation by rapidly warming in a 37° C. water bath. The cell suspension was diluted 1/10 into seeding medium (MatTek Corp.). The number of viable cells recovered (determined by trypan blue exclusion method) was determined by counting with a hemocytometer, and cells were centrifuged at 300×g for 10 minutes. The cell pellet was resuspended in EpiDerm' seeding medium (MatTek) at a density of 7.5×10⁵ cells/ml, and seeded in a volume of 0.4 ml directly onto a microporous polyethylene terephthalate (PET) membrane insert that had been precoated with collagen type 1 (Becton Dickenson, or equivalent) using methods that are common in the art.

For comparison, some inserts were also seeded with NHEK that had been expanded just prior to seeding by the standard procedure in the art. In this case, NHEK cells that had been cryopreserved at a density of 0.5×10⁶ cells/mL were recovered from cryopreservation by rapidly warming in a 37° C. water bath, diluted to a density of about 17,000 cells/mL into expansion medium (Medium 154, Life Technologies) and plated in p150 culture dishes (Corning) at a density of about 3,300 cells/cm². Cells were then cultured in a 37° C., 5% CO₂ incubator to allow cell expansion. Plates were fed with fresh expansion medium every 24-48 hrs until the cells were approximately 80% confluent (after 6 days). Cells were then harvested by trypsinization. For harvesting, plates were first rinsed with Ca²⁺/Mg²⁺ free phosphate buffered saline (PBS, Sigma), then incubated with a solution of 0.5% trypsin/ethylenediaminetetraacetic acid (EDTA) (Sigma) in PBS for approximately 10 minutes to cause detachment of the NHEK cells from the plate. Following cell detachment, trypsin was inactivated by addition of soybean trypsin inhibitor (Sigma). The suspension of detached cells was collected and centrifuged for 10 minutes at 300×g. The cell pellet was re-suspended in a small volume of EpiDerm^{TM} seeding medium (MatTek) and the number of cells obtained was determined by counting a small aliquot of the cell suspension (Coulter Counter). The final cell number was adjusted with seeding medium to 7.5×10⁵ cells/mL, and seeded in a volume of 0.4 mL directly onto a microporous polyethylene terephthalate (PET) membrane insert (MatTek Corp.) that had been precoated with collagen type 1 (Becton Dickenson).

The inserts that were prepared using both the standard and new direct seed methods were placed into a 24-well plate containing 0.5 mL/well of seeding medium per well, and the plate was then placed in a 37° C., 5% CO₂ incubator overnight to allow cell attachment to the insert membrane, and formation of a confluent cell monolayer. The seeding medium on both the top and underside of the culture inserts was then replaced 24 hours later with EpiDerm™ differentiation medium (MatTek Corp.) and culture was continued at 37° C., 5% CO₂ for 2 additional days.

Culture medium was then removed from the top surface of the insert membrane. The medium beneath the insert membrane was also replaced with fresh EpiDerm™ differentiation medium. Culture development was continued under these air-liquid interface (ALI) conditions in a 37° C., 5% CO₂ incubator for an additional 7 days, with feeding every 24-48 hours with fresh differentiation medium to produce the fully differentiated epidermal tissue.

Characterization of the tissues, including histological evaluation (FIG. 3) and barrier assessment (Table 1), demonstrated that the epidermal tissue produced by the direct seed method (“EPI-200 (Direct Seed)”) met all standard quality control specifications, and was fully equivalent to the EpiDerm™ tissues produced by the standard cell expansion procedure (“EPI-200 (Standard Method)”). These are representative data using cells derived from 1 individual donor. Similar results were obtained with cells derived from 2 additional donors (n=3 donors total).

TABLE 1
Barrier Assessment of 3D epidermal tissue equivalents (EPI-200)
produced via standard method versus direct seed process

| | ET-50¹ | | |
|---|---|---|---|
| Product | (hours) | Baseline Viability² | CV³ |
| EPI-200 (Standard Method) | 7.82 | 1.702 | 8.9 |
| EPI-200 (Direct Seed) | 8.27 | 1.633 | 17.5 |

¹Barrier determination by the ET-50 assay. The assay measures the time required for a topically applied solution of 1% Triton X-100 in water to penetrate the stratum corneum barrier of the tissue equivalent and reduce the viability of the tissue equivalent to 50% compared to water treated control equivalents. Tissue equivalent viability was determined by reduction of MTT (3-(4,5-Dimethylthiazol-2-yl)-2,5-Diphenyltetrazolium Bromide, Sigma) to form a purple formazan dye. MTT was added to the culture medium at a concentration of 1.0 mg/mL. Following incubation for 3 hours at 37° C., the dye was extracted from the tissue equivalents and quantified by measuring the optical density of the extracted dye solution at 595 nm.
²Baseline viability of the water treated control tissues as determined by the MTT viability assay.
³CV = coefficient of variation of the ET50 value.

### Example 2

### Production of 3D organotypic Skin Model Containing keratinocytes and melanocytes

Normal human epidermal keratinocytes and normal human dermal melanocytes were cryopreserved at a ratio of 10:1, and total density of 11×10⁶ cells/mL in medium consisting of 80% (v/v) Medium 154 (Life Technologies), 20% (v/v) fetal bovine serum (Hyclone) and 10% (v/v) dimethylsulfoxide (Sigma) using a controlled rate freezer (Cryomed). Cells were stored in the vapor phase of a liquid N₂ storage dewar.

Cells were recovered from cryopreservation by rapidly warming in a 37° C. water bath. The cell suspension was diluted 1/10 into MelanoDerm™ seeding medium (MatTek Corp.). The number of viable cells recovered (determined by trypan blue exclusion method) was determined by counting with a hemocytometer, and cells were centrifuged at 300×g for 10 minutes. The cell pellet was resuspended in MelanoDerm™ seeding medium at a density of 7.5×10⁵ cells/ml, and seeded in a volume of 0.4 mL directly onto a microporous modified Teflon™ membrane insert (Millipore) that had been precoated with collagen type 1 (Becton Dickinson, Trevigen or equivalent).

For comparison, some inserts were also seeded with NHEK and melanocytes that had been expanded just prior to seeding by the standard procedure in the art. In this case, NHEK cells that had been cryopreserved at a density of 0.5×10⁶ cells/mL were recovered from cryopreservation by rapidly warming in a 37° C. water bath, diluted to a density of about 17,000 cells/mL into NHEK expansion medium (Medium 154, Life technologies) and plated in p150 culture dishes (Corning) at a density of about 3,300 cells/cm². Cells were then cultured in a 37° C., 5% CO₂ incubator to allow cell expansion. Plates were fed with fresh NHEK expansion medium every 24-48 hrs until the cells were approximately 80% confluent (after 6 days). NHEKs were then harvested by trypsinization. For harvesting, plates were first rinsed with Ca²⁺/Mg²⁺ free PBS (Sigma), then incubated with a solution of 0.5% trypsin/EDTA (Sigma) in PBS for approximately 10 minutes to cause detachment of the NHEK cells from the plate. Following cell detachment, trypsin was inactivated by addition of soybean trypsin inhibitor (Sigma). The suspension of detached NHEK cells was collected and centrifuged for 10 minutes at 300×g. The NHEK cell pellet was re-suspended in a small volume of MelanoDerm™ seeding medium (MatTek) and the number of cells obtained was determined by counting a small aliquot of the cell suspension (Coulter Counter). Melanocytes that had been cryopreserved at a density of 300,000-600,000 cells/mL were recovered from cryopreservation by rapidly warming in a 37° C. water bath, diluted to a density of about 30,000-60,000 cells/mL into melanocyte expansion medium (NHM-GM, MatTek) and plated in p100 culture dishes (Corning) at a density of about 2500-5000 cells/cm². Cells were then cultured in a 37° C., 5% CO₂ incubator to allow cell expansion. Plates were fed with fresh melanocyte expansion medium every 24-48 hrs until the cells were approximately 80% confluent (after 11 days). Melanocytes were then harvested by trypsinization. For harvesting, plates were first rinsed with Ca²⁺/Mg²⁺ free PBS (Sigma), then incubated with a solution of 0.5% trypsin/EDTA (Sigma) in PBS for approximately 1 minute to cause detachment of the melanocytes from the plate. Following cell detachment, trypsin was inactivated by addition of NHM-GM (MatTek). The suspension of detached melanocytes was collected and centrifuged for 10 minutes at 300× g. The melanocyte cell pellet was re-suspended in a small volume of MelanoDerm™ seeding medium (MatTek) and the number of cells obtained was determined by counting a small aliquot of the cell suspension (Coulter Counter). The final number of NHEK and melanocytes was adjusted with seeding medium to 7.5×10⁵ cells/mL, and 0.75×10⁵, respectively, and seeded in a volume of 0.43 mL directly onto a microporous modified Teflon™ membrane insert (Millipore) that had been precoated with collagen type 1 (Becton Dickinson).

The inserts that were prepared using both the standard and new direct seed methods were placed into a 24-well plate containing 0.5 mL/well of MelanoDerm™ seeding medium per well. The inserts were incubated in 37° C., 5% CO₂ incubator overnight to allow cell attachment to the insert membrane, and formation of a confluent cell monolayer. The seeding medium on both the top and underside of the culture inserts was then replaced with MelanoDerm™ differentiation medium (MatTek) and culture was continued at 37° C., 5% CO₂ for 2 additional 2 days.

Culture medium was then removed from the top surface of the insert membrane. The medium beneath the insert membrane was also replaced 24 hours later with differentiation medium. Culture development was continued under these air-liquid interface (ALI) conditions in a 37° C., 5% CO₂ incubator for an additional 7 days to produce the fully differentiated MelanoDerm™ tissue.

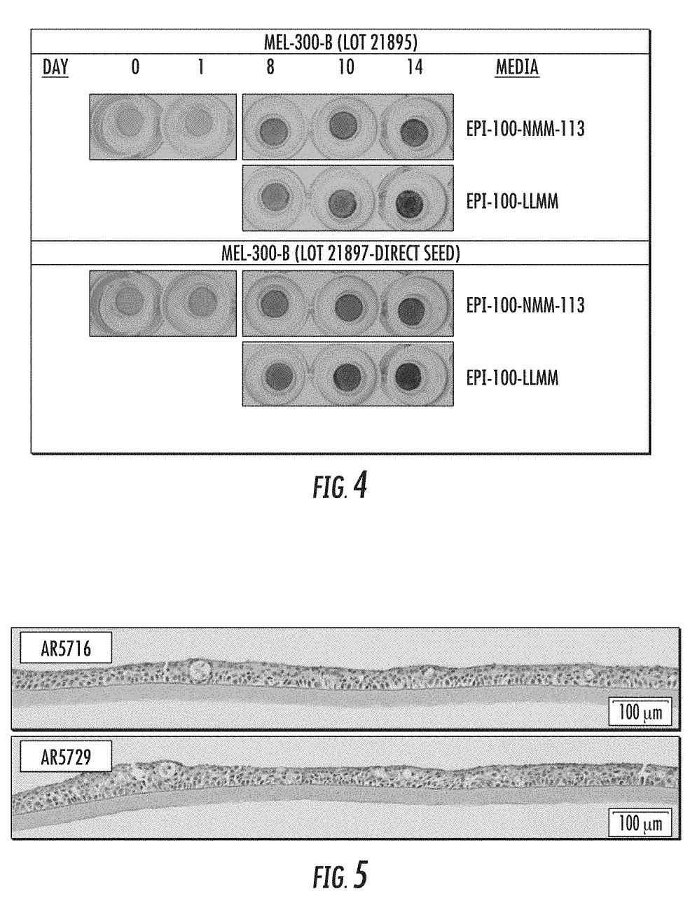

Evaluation of macroscopic pigmentation kinetics (FIG. 4) and barrier assessment (Table 2) demonstrated that the epidermal tissue produced by the direct seed method (“MEL-300-B (Direct Seed)”) met all standard quality control specifications, and was fully equivalent to the MelanDerm™ tissues produced by the standard cell expansion procedure (“MEL-300-B (Standard Method)”).

TABLE 2
Barrier Assessment of 3D organotypic skin equivalent model
(MEL-300-B) produced via cell expansion versus direct seed

| | Viability 5 hr | Baseline | |
|---|---|---|---|
| | post Triton X-100 | Viability² | |
| Product | treatment¹ (%) | (OD₅₉₅) | CV³ |
| MEL-300-B (Standard Method) | 86.4 | 1.692 | 3.6 |
| MEL-300-B (Direct Seed) | 91.9 | 1.667 | 2.1 |

¹Barrier determination by the MTT viability assay. The assay measured the viability 5 hours after treatment with a topically applied solution of 1% Triton X-100 in water. The Triton X-100 penetrates the stratum corneum barrier and reduces the viability of the tissue equivalent_compared to water treated control tissues. Tissue viability was determined by reduction of MTT (3-(4,5-Dimethylthiazol-2-yl)-2,5-Diphenyltetrazolium Bromide, Sigma) to form a purple formazan dye. MTT was added to the culture medium at a concentration of 1.0 mg/mL. Following incubation for 3 hours at 37° C., the dye was extracted from the tissue equivalents and quantified by measuring the optical density of the extracted dye solution at 595 nm.
²Baseline viability of the water treated control tissues as determined by the MTT viability assay.
³CV = coefficient of variation of the 5 hr % viability value.

### Example 3

### Production of 3D organotypic Airway Tissue Equivalent Model Containing bronchial epithelial Cells

Normal human bronchial epithelial cells (NHBE) were cryopreserved at a density of 1×10⁷ cells/mL in medium consisting of 80% (v/v) NHBE growth medium (MatTek), 10% (v/v) fetal bovine serum (Hyclone) and 10% (v/v) dimethylsulfoxide (Sigma) using a controlled rate freezer (Cryomed). Cells were stored in the vapor phase of a liquid N₂ storage dewar.

Cells were recovered from cryopreservation by rapidly warming in a 37° C. water bath. The cell suspension was diluted 1/10 into NHBE growth medium (MatTek, Lonza or equivalent). The number of viable cells recovered (determined by trypan blue exclusion method) was determined by counting with a hemocytometer, and cells were centrifuged at 300×g for 10 minutes. The cell pellet was resuspended in EpiAirway™ seeding medium at a density of 0.5×10⁶ cells/mL, and seeded in a volume of 0.4 ml directly onto a microporous polycarbonate membrane insert (Millipore) that had been precoated with collagen type 1 (Becton Dickinson).

For comparison, some inserts were also seeded with NHBE that had been expanded just prior to seeding by the standard procedure in the art. In this case, NHBE cells that had been cryopreserved at a density of 0.5×10⁶ cells/mL were recovered from cryopreservation by rapidly warming in a 37° C. water bath, diluted to a density of about 17,000 cells/mL into NHBE expansion medium (MatTek NHBE-GM) and plated in p150 culture dishes (Corning) at a density of about 3,300 cells/cm². Cells were then cultured in a 37° C., 5% CO₂ incubator to allow cell expansion. Plates were fed with fresh NHBE expansion medium every 24-48 hrs until the cells were approximately 80% confluent (generally 5-6 days). Cells were then harvested by trypsinization. For harvesting, plates were first rinsed with Ca²⁺/Mg²⁺ free PBS (Sigma), then incubated with a solution of 0.5% trypsin/EDTA (Sigma) in PBS for approximately 10 minutes to cause detachment of the NHBE cells from the plate. Following cell detachment, trypsin was inactivated by addition of trypsin inhibitor (Sigma). The suspension of detached cells was collected and centrifuged for 10 minutes at 300×g. The cell pellet was re-suspended in a small volume of EpiAirway™seeding medium (MatTek) and the number of cells obtained was determined by counting a small aliquot of the cell suspension (Coulter Counter). The final cell number was adjusted with seeding medium to 5.0×10⁵ cells/mL, and seeded in a volume of 0.4 mL directly onto a microporous polycarbonate membrane insert (Millipore) that had been precoated with collagen type 1 (Becton Dickinson).

The inserts prepared using the standard and new direct seed methods were placed into a 24-well plate containing 0.5 ml/well of seeding medium (MatTek Corp.). The inserts were incubated in 37° C., 5% CO₂ incubator overnight to allow cell attachment to the insert membrane. The seeding medium on both the top and underside of the culture inserts was then replaced with airway differentiation medium (MatTek) and culture was continued at 37° C., 5% CO₂ for 2 additional days.

Culture medium was then removed from the top surface of the insert membrane. The medium beneath the insert membrane was also replaced with differentiation medium. Culture development was continued under these air-liquid interface (ALI) conditions in a 37° C., 5% CO₂ incubator for an additional 20 days, with feeding every 24-48 hours with fresh EpiAirway™ differentiation medium to produce the fully differentiated airway tissue.

Histological evaluation (FIG. 5), barrier assessment (Table 3) and functional characterization demonstrated that the bronchial epithelial tissue produced by the direct seed method met all standard quality control specifications, and was fully equivalent to the EpiAirway™ tissues produced by the standard cell expansion procedure.

TABLE 3
Barrier Assessment of 3D organotypic airway tissue
equivalent model (EpiAirway ™) produced via
standard method with cell expansion versus direct seed

| | Transepithelial Electrical | |
|---|---|---|
| | Resistance (TEER) | Standard |
| Method | (Ω × cm²) | Deviation |
| Standard Method | 339.7 | 42.3 |
| Direct Seed | 369.2 | 98.5 |

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention.

All publications, patents, and patent applications cited herein are hereby incorporated by reference in their entireties for all purposes and to the same extent as if each individual publication, patent, or patent application were specifically and individually indicated to be so incorporated by reference.

We claim:

1. A method for producing a tissue equivalent, comprising:
(a) seeding frozen cryopreserved undifferentiated cells directly onto a support immediately following recovery from cryopreservation; and
(b) culturing the cells in contact with a differentiation medium and under conditions appropriate for differentiation.

2. A method according to claim 1, wherein recovery from cryopreservation comprises rapidly thawing the cells and diluting the thawed cell suspension into culture medium.

3. A method according to claim 1, wherein the differentiation medium comprises at least about 200 μM calcium and growth and differentiation factors for differentiation of the cells into a desired tissue.

4. A method according to claim 1, wherein between steps (a) and (b), the cells are cultured in contact with a propagation medium and under conditions appropriate for propagation, and wherein the propagation medium is removed prior to addition of the differentiation medium in step (b).

5. A method according to claim 4, wherein the propagation medium comprises less than about 200 μM calcium and growth factors and other components for propagation of the undifferentiated cells.

6. A method according to claim 4, wherein the cells are incubated in contact with the propagation medium for about 2 hours to about 7 days to achieve confluence of cell monolayer prior to removal and replacement of the propagation medium with differentiation medium.

7. A method according to claim 6, wherein the cells differentiate after contact with the differentiation medium for about 7 days to about 28 days.

8. A method according to claim 1, wherein the cryopreserved cells comprise a density of about 1×10⁶ to about 20×10⁶ per mL in cryopreservation medium.

9. A method according to claim 8, wherein the cryopreservation medium comprises one or more of serum, bovine serum albumin (BSA), glycerol, and dimethylsulfoxide (DMSO).

10. A method according to claim 9, wherein the cryopreservation medium comprises a pH of about 6.8 to about 7.8.

11. A method according to claim 9, wherein the cryopreservation medium comprises osmolality of about 250 to about 350 mOsm/kg.

12. A method according to claim 1, wherein the cryopreserved cells are seeded directly onto the support at a density of about 100×10³ to about 1500×10³ per cm².

13. A method according to claim 1, wherein the support comprises a microporous membrane through which culture medium may pass.

14. A method according to claim 13, wherein the membrane is coated with extracellular matrix material.

15. A method according to claim 14, wherein the extracellular matrix material comprises one or more of an attachment factor, a cell surface integrin, collagen I, III, or IV, fibronectin, laminin, hyaluronic acid, and a growth factor.

16. A method according to claim 15, wherein the attachment factor comprises laminin, entactin, collagen and/or heparin sulfate proteoglycans.

17. A method according to claim 15, wherein the growth factor comprises transforming growth factor (TGF)-beta and/or epidermal growth factor (EGF).

18. A method according to claim 13, wherein the support is in the form of an insert that fits within a well of a multiwell plate.

19. A method according to claim 13, wherein the support is in the form of a high throughput screening (HTS) plate.

20. A method according to claim 13, wherein the membrane comprises polycarbonate, polyester, modified polytetrafluorethylene, microporous silica, ceramic material, or cellulose nitrate.

21. A method according to claim 1, wherein the cells comprise undifferentiated skin cells.

22. A method according to claim 21, wherein the skin cells comprise keratinocytes, melanocytes, or a mixture of keratinocytes and melanocytes.

23. A method according to claim 22, wherein the skin cells comprises a mixture of keratinocytes and melanocytes in a ratio of about 3:1 to about 30:1.

24. A method according to claim 1, wherein the cells comprise one or more of undifferentiated keratinocytes, melanocytes, fibroblasts, endothelial cells, airway epithelial cells, corneal epithelial cells, vaginal epithelial cells, oral epithelial cells, intestinal epithelial cells, dendritic cells, macrophages, kidney cells, liver cells, neuronal cells, and mast cells.

25. A method according to claim 24, wherein the cells comprise human cells.

26. A kit for producing a tissue equivalent, comprising:
(a) frozen cryopreserved undifferentiated cells;
(b) a support for growth of the cells; and
(c) one or more culture media for propagation and/or differentiation of the cells.

27. A kit according to claim 26, further comprising instructions directing seeding of the cells directly onto the support immediately following recovery from cryopreservation without additional steps for expansion, harvesting, counting, and/or dilution of the cells beforehand.

28. A kit according to claim 26, wherein the cryopreserved cells comprise a density of about 1×10⁶ to about 20×10⁶ per mL in cryopreservation medium.

29. A kit according to claim 28, wherein the cryopreservation medium comprises one or more of serum, BSA, glycerol, and DMSO.

30. A kit according to claim 26, wherein the support comprises a microporous membrane through which culture medium may pass.

31. A kit according to claim 30, wherein the membrane is coated with extracellular matrix material.

32. A kit according to claim 31, wherein the extracellular matrix material comprises one or more of an attachment factor, a cell surface integrin, collagen I, III, or IV, fibronectin, laminin, hyaluronic acid, and a growth factor.

33. A kit according to claim 30, wherein the support is in the form of an insert that fits within a well of a multiwell plate or in the form of a HTS plate.

34. A kit according to claim 30, wherein the membrane comprises polycarbonate, polyester, modified polytetrafluorethylene, microporous silica, ceramic material, or cellulose nitrate.

35. A kit according to claim 26, wherein the cells comprise undifferentiated skin cells.

36. A kit according to claim 35, wherein the skin cells comprise keratinocytes, melanocytes, or a mixture of keratinocytes and melanocytes.

37. A kit according to claim 26, wherein the cells comprise one or more of undifferentiated keratinocytes, melanocytes, fibroblasts, endothelial cells, airway epithelial cells, corneal epithelial cells, vaginal epithelial cells, oral epithelial cells, intestinal epithelial cells, dendritic cells, macrophages, kidney cells, liver cells, neuronal cells, and mast cells.

38. A kit according to claim 37, wherein the cells comprise human cells.
